(19) United States
(12) Patent Application Publication
(10) Pub. No.: US 2022/0202737 A1
(43) Pub. Date: Jun. 30, 2022

(54) TREATMENT OF HIDRADENITIS SUPPURATIVA WITH TAPINAROF COMPOSITIONS

(71) Applicant: Sol-Gel Technologies Ltd., Ness Ziona (IL)

(72) Inventor: Moshe ARKIN, Kfar Shmaryahu (IL)

(73) Assignee: Sol-Gel Technologies Ltd., Ness Ziona (IL)

(21) Appl. No.: 17/598,271

(22) PCT Filed: Mar. 26, 2020
(86) PCT No.: PCT/IL2020/050369

§ 371 Date: Sep. 26, 2021

Related U.S. Application Data
(60) Provisional application No. 62/823,886, filed on Mar. 26, 2019.

(51) Int. Cl. A61K 31/05; A61K 9/00; A61K 31/235; A61K 45/06

(52) U.S. Cl. CPC A61K 31/05; A61K 45/06; A61K 31/235; A61K 9/0014

(57) ABSTRACT

The present invention, in some embodiments thereof, relates to treatment of hidradenitis suppurativa by topical or intralesional administration of a composition comprising tapinarof and optionally at least one additional active agent. The composition of the present invention is useful for the treatment, prevention or amelioration of hidradenitis suppurativa.

### CROSS-REFERENCE TO RELATED APPLICATIONS

This application is a National Phase Application of PCT International Application No. PCT/IL2020/050369, International Filing Date Mar. 26, 2020, claiming the benefit of U.S. Patent Application No. 62/823,886, filed Mar. 26, 2019, which is hereby incorporated by reference.

### FIELD OF THE INVENTION

The present invention, in some embodiments thereof, relates to treatment of hidradenitis suppurativa by administering a composition comprising tapinarof and optionally at least one additional active agent. The composition of this invention is useful for the treatment, prevention or amelioration of hidradenitis suppurativa.

### BACKGROUND

Hidradenitis suppurativa (HS), also known as acne inversa, is a long-term chronic skin disease whose present treatment options are often unsatisfactory. HS has a profound effect on patient's quality of life (QoL). Alavi A. et al., reviewed QoL aspects of this disease in an article titled “Quality-of-Life Impairment in Patients with Hidradenitis Suppurativa” (Am J Clin Dermatol, 2014, vol. 15. No. 6).

The clinical picture of HS includes solitary nodules, diffuse, painful abscesses, malodorous drainage, sinus tract formation and scarring.

The exact cause of hidradenitis suppurativa is still unclear, but it is believed that the underlying mechanism involves dysfunction of the apocrine sweat glands or hair follicles.

There is no cure for HS, but treatments with drugs selected from oral antibiotics, corticosteroid injections, antiandrogen therapy with high dosages of cyproterone acetate and ethynyl estradiol, TNF inhibitors like adalimumab and immunosuppressive drugs have been attempted.

Tapinarof (3,5-dihydroxy-4-isopropyl-trans-stilbene) is a pharmaceutical active agent investigated for the treatment of atopic dermatitis, psoriasis and psoriatic disorders (Zang Y N, et al., Int J Clin Pharmacol Ther. 2016 February; 54(2):87-95). The 3,5-dihydroxy-4-isopropyl-stilbene is also known as benvitimod.

Tapinarof is a first-in-class drug, whose mechanism is not yet fully understood.

### SUMMARY OF THE INVENTION

This invention provides a composition and methods of treatment of hidradenitis suppurativa by administration to a subject in need thereof a composition comprising from about 0.25% w/w to about 10.0% w/w tapinarof and a carrier. In another embodiment, the composition is formulated for topical, transdermal or intralesional administration.

This invention provides a topical composition and methods of treatment of hidradenitis suppurativa by administration to a subject in need thereof a topical composition comprising from about 0.25% w/w to about 10.0% w/w tapinarof and a carrier suitable for topical administration.

Also provided is a combination composition comprising from about 0.25% w/w to about 10.0% w/w tapinarof, at least one additional active agent selected from about 0.05% w/w to about 20% w/w at least one additional active agent selected from an antibiotic, a retinoid, azelaic acid, benzoyl peroxide and combinations thereof, and a carrier suitable for topical or intralesional administration.

The present invention also provides dosage forms, kits comprising the above compositions.

The compositions, dosage forms, kits and methods of this invention are suitable for the treatment, prevention or alleviation of hidradenitis suppurativa and exhibit synergistic and/or additive effects which allow reducing the amounts of the active agents in the compositions.

### DETAILED DESCRIPTION OF THE INVENTION

### Tapinarof Compositions for Treatment of Hidradenitis Suppurativa

In one embodiment, the present invention provides compositions, kits and articles of manufacture that include tapinarof, optionally in combination with at least one additional active agent, for treatment of hidradenitis suppurativa. The compositions and articles of manufacture can be administered using a variety of routes such as topical application, transdermal application or intralesional administration including intralesional injection. The preferred route is the topical route and the preferred formulations are the cream and the foam and the lotion.

In one embodiment, the present invention provides methods for the treatment, prevention and/or amelioration of hidradenitis suppurativa comprising a composition comprising from about 0.25% w/w to about 10% w/w tapinarof. In another embodiment, the composition comprises from 0.25% w/w to 0.5% w/w tapinarof. In another embodiment, the composition comprises from 0.5% w/w to 1% w/w tapinarof. In another embodiment, the composition comprises from 1% w/w to 1.5% w/w tapinarof. In another embodiment, the composition comprises from 1.5% w/w to 2% w/w tapinarof. In another embodiment, the composition comprises from 2% w/w to 5% w/w tapinarof. In another embodiment, the composition comprises from 5% w/w to 10% w/w tapinarof. Each possibility represents a separate embodiment of the present invention. In another embodiment, the composition is a topical composition. In another embodiment, the composition is an intralesional injection.

A number of active agents selected from oral antibiotics, corticosteroid injections, antiandrogen therapy with high dosages of cyproterone acetate and ethynyl estradiol, TNF inhibitors like adalimumab and immunosuppressive drugs have been tried in HS treatments.

One of the topical drugs used so far is isotretinoin, but it was found ineffective when used as a single drug.

In one embodiment, the present invention also provides a combination composition comprising from about 0.25% w/w to about 10.0% w/w tapinarof, from about 0.05% w/w to about 20.0% w/w at least one additional active agent selected from an antibiotic, antiandrogen (e.g. cyproterone acetate), estrogen (e.g. ethynyl estradiol), tumor necrosis factor (TNF) inhibitor (e.g. adalimumab), a retinoid, azelaic acid, benzoyl peroxide and combinations thereof; and a carrier suitable for topical and intralesional administration. Each possibility represents a separate embodiment of the present invention. In another embodiment, the composition is topical. In another embodiment, the composition is intralesional.

In one embodiment, the present invention also provides a topical combination composition comprising from about 0.25% w/w to about 10.0% w/w tapinarof, from about 0.05% w/w to about 20.0% w/w at least one additional active agent selected from a topical antibiotic, antiandrogen (e.g. cyproterone acetate), estrogen (e.g. ethynyl estradiol), tumor necrosis factor (TNF) inhibitor (e.g. adalimumab), a topical retinoid, azelaic acid, benzoyl peroxide and combinations thereof; and a carrier suitable for topical administration. Each possibility represents a separate embodiment of the present invention.

In another embodiment, the compositions of this invention comprise from 0.25% w/w to 0.5% w/w tapinarof. In another embodiment, the composition comprises from 0.5% w/w to 1% w/w tapinarof. In another embodiment, the composition comprises from 1% w/w to 1.5% w/w tapinarof. In another embodiment, the composition comprises from 1.5% w/w to 2% w/w tapinarof. In another embodiment, the composition comprises from 2% w/w to 5% w/w tapinarof. In another embodiment, the composition comprises from 5% w/w to 10% w/w tapinarof. Each possibility represents a separate embodiment of the present invention. In another embodiment, the composition comprises from 0.05% w/w to 0.1% w/w additional active agent. In another embodiment, the composition comprises from 0.1% w/w to 0.25% w/w additional active agent. In another embodiment, the composition comprises from 0.25% w/w to 0.5% w/w additional active agent. In another embodiment, the composition comprises from 0.5% w/w to 1% w/w additional active agent. In another embodiment, the composition comprises from 1% w/w to 1.5% w/w additional active agent. In another embodiment, the composition comprises from 1.5% w/w to 2% w/w additional active agent. In another embodiment, the composition comprises from 2% w/w to 5% w/w additional active agent. In another embodiment, the composition comprises from 5% w/w to 10% w/w additional active agent. In another embodiment, the composition comprises from 10% w/w to 15% w/w additional active agent. In another embodiment, the composition comprises from 15% w/w to 20% w/w additional active agent. Each possibility represents a separate embodiment of the present invention.

In another embodiment, the at least one antibiotic in the compositions of this invention is selected from clindamycin, gentamicin, w/w erythromycin, a quinolone and combinations thereof. In another embodiment, the combination composition comprising tapinarof, at least one antibiotic and a suitable carrier is a formulated to be administered topically. In another embodiment, the combination composition comprising tapinarof, at least one antibiotic is a formulated to be administered intralesional.

In another embodiment, the at least one retinoid in the compositions of this invention is selected from acitretin, retinol, tazarotene, tretinoin, isotretinoin and combinations thereof. In another embodiment, the combination composition comprising tapinarof, at least one retinoid and a suitable carrier is a formulated to be administered topically. In another embodiment, the combination composition comprising tapinarof, at least one antibiotic is a formulated to be administered intralesional.

In some embodiments, tapinarof and optionally at least one additional active agent in the compositions of this invention are included in an amount effective for treating, preventing or reducing the hidradenitis suppurativa symptoms. The concentrations of the active agents in the composition will depend on absorption, inactivation, excretion rates of the active compound, the synergistic or additive effects, the dosage schedule, and amount administered as well as other factors known to those of skill in the art. Generally, the dosages and concentrations will be lower, typically at least about or at 5 to 10% lower but up to about 15%, 20%, 25%, 30%, 35%, 40%, 50%, 90% or 95% lower than the amount of tapinarof and optionally at least one additional active agent in the marketed single drug currently administered or considered for the treatment of hidradenitis suppurativa. The dosage and regimen of administration may be determined by dose finding studies, as known in the art. Each possibility represents a separate embodiment of the present invention.

Exemplary dosages, strengths and concentrations of tapinarof in the tapinarof compositions administered topically, can be in the range of from about or at 0.1%, 0.25%, 0.5%, 1%, 2%, 3%, 4%, 5%, 6%, 7%, 8%, 9% or 10% w/w. Typical strengths in the topical combination compositions of this invention are 0.25%, 0.5%, 1% or 2% w/w tapinarof. The frequency of administration can be determined empirically. Exemplary frequencies are once daily, twice daily, weekly, bi-weekly or monthly. Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the hidradenitis suppurativa. For example, dosage administration can begin at from twice a day, to once a day, to two times a week, to once a week, to once every two weeks or less frequent than once every two weeks.

Pharmaceutical carriers or vehicles suitable for preparation of the compositions provided herein include any such carriers known to those skilled in the art to be suitable for the particular mode of administration.

The resulting composition may be a lotion, a solution, a suspension, an emulsion or the like and is formulated as creams, gels, ointments, emulsions, solutions, elixirs, lotions, suspensions, tinctures, pastes, foams, aerosols, sprays, patches, foams, or any other formulation suitable for topical administration. The preferred compositions are the cream, the foam and the lotion.

The resulting composition may be administered intralesional. In another embodiments the composition is an intralesional injection or microneedles.

Pharmaceutical carriers or vehicles suitable for administration of the active agents provided herein include any such carriers known to those skilled in the art to be suitable for the particular mode of administration. In addition, the active agents may be formulated as the sole pharmaceutically active agent in the composition or may be combined with other active agents. The active agents are included in the carrier in an amount sufficient to exert a therapeutically useful effect i.e., amelioration of the symptoms of the hidradenitis suppurativa, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include those suited for use include lotions, creams, solutions, gels, foams and the like. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

The compositions according to the invention are pharmaceutical compositions, and especially dermatological compositions, which may be in any form conventionally used for topical application and especially in the form of lotions, creams. By addition of a fatty or oily phase, it may also be in the form of dispersions of the lotion or serum type, emulsions of liquid or semi-liquid consistency of the milk type obtained by dispersing a fatty phase in an aqueous phase (O/W) or conversely (W/O), or suspensions or emulsions of soft, semi-liquid or solid consistency of the cream, gel or ointment type, or alternatively multiple emulsions (W/O/W or O/W/O), microemulsions, microcapsules, microparticles or vesicular dispersions of ionic and/or nonionic type, or wax/aqueous phase dispersions.

The composition according to the invention is an intralesional composition. In another embodiment, intralesional administration is done by regular injections or with microneedles. In another embodiment, the intralesional composition is delivered directly to a HS lesion on the skin. The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents. Such sterile formulations may be prepared using a non-toxic parenterally-acceptable diluent or solvent, such as water or 1,3-butane diol, for example. Other acceptable diluents and solvents include, but are not limited to, Ringers solution, isotonic sodium chloride solution, and fixed oils such as synthetic mono- or di-glycerides. Pharmaceutical compositions that are useful in the methods of the invention may be administered, prepared, packaged, and/or sold in formulations suitable for intralesional delivery. The compositions may contain pharmaceutically-acceptable carriers and other ingredients known to enhance and facilitate drug administration. Other possible formulations, such as nanoparticles, liposomes may also be used.

In further embodiments, a composition of the invention comprising, as a single pharmaceutical active agent, tapinarof in a solid form, for topical use in the treatment of hidradenitis suppurativa.

In further embodiments, a composition of the invention comprising, as a single pharmaceutical active agent, tapinarof, for intralesional injection use in the treatment of hidradenitis suppurativa.

In other embodiments, said composition further comprises at least one fatty alcohol.

In yet other embodiments, said composition further comprises a polyacrylic acid homopolymer or copolymer.

In some embodiments, said water in said oil in water emulsion further comprises at least one water soluble humectant.

In other embodiments, said at least one water soluble humectant is selected from the group consisting of propylene glycol, glycerin, and polyethylene glycol-X, where X is in the range of 200 to 10,000. Each possibility represents a separate embodiment of the present invention.

In some embodiments, the composition of the present invention comprises tapinarof and at least one additional active agent, where said additional active agent is described hereinabove. In another embodiment, the additional active agent is benzoyl peroxide. In another embodiment, the benzoyl peroxide is encapsulated. In another embodiment, tapinarof and the additional active agent are both encapsulated in the same capsule. In another embodiment, tapinarof and the additional active agent are both encapsulated in different capsules. In some embodiments, tapinarof and/or the additional active agent are each encapsulated in a microcapsule, where “microcapsule” refers to a microparticle having a core shell structure, wherein said core comprises an active agent as defined herein (e.g. tapinarof or benzoyl peroxide), being coated by a shell forming the microcapsule entrapping the core.

In further embodiments, the compositions of this invention for treating hidradenitis suppurativa are controlled or slowed release drug delivery system, wherein the active agent is encapsulated, coated, adsorbed, embedded, impregnated, dispersed, entrapped, or encased in a polymeric material and providing a sustained release formulation. In another embodiment, the one additional active agent is benzoyl peroxide and is encapsulated.

According to some embodiments of the present invention, the coated form of the benzoyl peroxide (microcapsule) may be in form of a polymeric microsponge/silica microsphere where the benzoyl peroxide is adsorbed, embedded, impregnated or entrapped in the microsponge/silica microsphere as described for example in U.S. Pat. Nos. 4,690,825; 5,145,675, 5,879,716, 5,955,109, and 9,452,137 incorporated herein by reference in their entirety.

In other embodiments, microcapsules are formed by the encapsulation process disclosed in the following publications (herein incorporated by reference): U.S. Pat. Nos. 7,629,394, 9,205,395, US 2015/0328615, US 2014/0186630. Controlled release microcapsules: IN01958CH2007, IN02080CH2007, U.S. Pat. Nos. 4,235,872, 4,670,250, EP 0248531, U.S. Pat. Nos. 4,970,031, 5,238,714, WO9321764, U.S. Pat. No. 5,575,987, WO9420075, US 2004/137031, US 2006/003014, US 2010/180464.

When referring to a “controlled or slowed release drug delivery system” it should be understood to relate to a delivery system (which in the present invention is a topical delivery system) that enables the release of the pharmaceutical active agent in predetermined amounts over a specified period. In some embodiments said system is a core-shell system of a microcapsule or a porous matrix structure, such as for example a microsponge.

The term “embedded” should be understood to encompass an inert system that provides a barrier between the pharmaceutical active agent, i.e. tapinarof, and its surrounding environment in the composition. In some embodiments said agent is entrapped and/or encapsulated in said controlled release system.

### Microcapsules

As used herein, the term “microcapsule” refers to any micro- or nano-sized particle having a core-shell structure that is capable of encasing, encapsulating or entrapping compounds, including but not limited to active ingredients such as BPO. In some embodiments, microcapsules are made by a sol-gel process, e.g., as generally described in WO 03/034979 and WO 2011/080741.

### Core

As used herein, the term “core” refers to the inside part of a microcapsule comprising at least one active ingredient surrounded by a shell of the microcapsule. In some embodiments, the core can be solid at room temperature. In other embodiments, the core can be in a semi-solid phase at room temperature. In some embodiments, the core can be in the form of an emulsion, for example an oil-in-water emulsion. In some embodiments, the core can be in the form of oil solution. In some embodiments, the core can be in the form of an aqueous solution. In some embodiments, the core can be in the form of a dispersion.

Additional compound(s) can be present in the core. Non-limiting examples of the additional compounds that can be present in the core include phase changing materials (PCMs), carriers, excipients, antioxidants, pharmaceutically acceptable polymers, and salts. In some embodiments, the core comprises at least one phase changing material. Exemplary phase changing materials include, but are not limited to, natural and synthetic paraffins; C₁₀-C₁₀₀ (straight, branched, and cyclic) alkanes, alkenes and alkynes; C₁₀-C₁₀₀ aliphatic alcohols (e.g., fatty alcohols); fatty acids; carnauba wax; beeswax; and mixtures thereof. In some embodiments, the core comprises at least one antioxidant. Examples of antioxidants include, but are not limited to, butylated hydroxytoluene (BHT), butylated hydroxyanisole (BHA), vitamin E, vitamin E acetate, vitamin E palmitate, vitamin C, an ester of vitamin C, and one or more salts of vitamin C.

### Shell

As used herein, the term “shell” refers to the part of a microcapsule that surrounds the core of the microcapsule. In some embodiments, the shell comprises an inorganic polymer (for example, a silica polymer). In some embodiments, the inorganic polymer can be prepared from a sol-gel precursor.

As used herein, the term “sol-gel precursor” refers to any metal or semi-metal organo-metallic monomer, or a prepolymer (which means several monomers polymerized together) thereof, which provide a glass or ceramic material by in-situ polymerization (an inorganic sol-gel polymerization process). In some embodiments, the sol-gel precursor can be a metal or semi-metal organo-metallic monomer. Examples of sol-gel precursor include, but are not limited to, a metal alkoxide monomer; a semi-metal alkoxide monomer; a metal ester monomer; a semi-metal ester monomer; a silazane monomer; a colloidal silica; a monomer of the formula M(R)ₙ(P)ₘ, where M can be a metallic or a semi-metallic element, R can be a hydrolyzable substituent, n can be an integer from 2 to 6, P can be a non polymerizable substituent, and m can be an integer from 0 to 6; and a partially hydrolyzed and partially condensed polymer thereof. Various metallic or semi metallic elements can be used in the sol-gel precursor, for example, Si, Ti, Zr, Al, and Zn. Examples of semi-metal alkoxide monomers include, but are not limited to, tetramethoxysilane (also known as tetramethyl orthosilicate or TMOS), tetraethoxysilane (also known as tetraethyl orthosilicate or TEOS), dimethyldimethoxysilane, methyltrimethoxysilane, diethyldimethoxysilane, and sodium silicate.

In some embodiments, the sol-gel precursor can be selected from a silicon alkoxide monomer; a silicon ester monomer; a monomer of the formula Si(R)ₙ(P)ₘ, wherein R can be a hydrolyzable substituent, n can be an integer from 2 to 4, P can be a non polymerizable substituent, and m can be an integer from 0 to 4; a partially hydrolyzed and partially condensed polymer of any of the above, and mixtures of any of the above. Non-limiting examples of silicon alkoxide monomer include tetramethoxy silane, tetraethoxy silane, and combinations thereof. Non-limiting examples of monomers of the formula Si(R)ₙ(P)ₘ include methyl trimethoxysilane, dimethyl dimethoxysilane, and combinations thereof.

In one embodiment, the first composition comprises a first core-shell microcapsules comprising a first core that comprises benzoyl peroxide and a first shell that comprises a first silica polymer, the benzoyl peroxide being present in the composition in an initial amount of about 10% by weight, based on the total weight of the first composition.

### Methods of Treatment

According to an aspect of the invention, there is provided a method of treatment of hidradenitis suppurativa comprising administering to a subject in need thereof a therapeutically effective amount of a composition comprising tapinarof and optionally at least one additional active agent.

In some embodiments, the therapeutically effective amount is an effective amount of tapinarof and at least one additional active agent, namely an amount which will cure, treat, mitigate and/or prevent hidradenitis suppurativa.

In some embodiments, co-administration of tapinarof and at least one additional active agent exhibits an additive and/or synergistic effect while treating, preventing or alleviating hidradenitis suppurativa.

In some other embodiments, the co-administration may be made either by administration of a single combination composition, or alternatively by separate administration of a first composition comprising tapinarof and a second composition comprising at least one additional active agent of this invention.

### Regimen of Administration of Topical Tapinarof Compositions for Treatment of Hidradenitis Suppurativa

Therapeutically effective concentrations of tapinarof and optionally at least one additional active agent in the compositions of this invention for treatment, prevention or amelioration of the symptoms manifested by hidradenitis suppurativa are determined by empirical methods known in the art.

The concentration of the active agents in the composition will depend on absorption, inactivation, excretion rates of the active compound, synergistic and/or additive effects, the dosage schedule, and amount administered as well as other factors known to those of skill in the art. Generally, the dosages and concentrations will be lower, typically at least about or at 5% to 10% lower but up to about or at 15%, 20%, 30%, 35%, 50%, 90% or 95% lower than the amount of tapinarof and at least one additional active agent in the developed or marketed single drug currently being developed or used for the treatment of hidradenitis suppurativa. The dosage and regimen of administration may be determined by dose finding studies, as known in the art.

Exemplary dosages, strengths and concentrations of tapinarof in the tapinarof compositions administered topically or intralesional, can be in the range of from about or 0.1%, 0.25%, 0.5%, 1%, 2%, 3%, 4%, 5%, 6%, 7%, 8%, 9% or 10% w/w.

Typical strengths in the topical combination compositions of this invention are 0.25%, 0.5%, 1% or 2% w/w tapinarof.

The frequency of administration can be determined empirically. Exemplary frequencies are once daily, twice daily, weekly, bi-weekly or monthly.

Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of hidradenitis suppurativa. For example, dosage administration can begin at from twice a day, to once a day, to two times a week, to once a week, to once every two weeks or less frequent than once every two weeks.

### Kits

There are provided kits containing the compositions of this invention, optionally including instructions for administration. The combinations include, for example, the compositions as provided herein. Additionally, provided herein are kits containing the above-described combinations and optionally instructions for administration by topical, transdermal, intralesional or other routes, depending on the composition to be delivered.

The compositions provided herein can be packaged as articles of manufacture containing packaging material, a composition provided herein, and a label that indicates that the composition is for treating hidradenitis suppurativa, and is formulated for topical or intralesional delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes and any packaging material suitable for a selected formulation and intended mode of administration and treatment.

### Embodiments

In some embodiments, there is provided a composition for the treatment, prevention and/or amelioration of hidradenitis suppurativa, comprising from about 0.25% w/w to about 10.0% w/w tapinarof and a carrier.

In another embodiment, the composition is administered topically, transdermally or intralesionally.

In some embodiments, there is provided a topical composition for the treatment, prevention and/or amelioration of hidradenitis suppurativa, comprising from about 0.25% w/w to about 10.0% w/w tapinarof and a carrier suitable for topical administration.

In some other embodiments, there is provided a composition comprising from about 0.25% w/w to about 10.0% w/w tapinarof, from about 0.05% w/ to about 20% w/w at least one additional active agent selected from an antibiotic, a retinoid, azelaic acid, benzoyl peroxide and combinations thereof and a carrier for topical or intralesional administration.

In some other embodiments, there is provided a composition comprising from about 0.25% w/w to about 10.0% w/w tapinarof, from about 0.05% w/ to about 20% w/w at least one additional active agent selected from a topical antibiotic, a topical retinoid, azelaic acid, benzoyl peroxide and combinations thereof and a carrier suitable for topical administration.

In some embodiments, the composition of this invention comprises at least one additional active agent. In another embodiment, the additional active agent is an antibiotic selected from about 1% w/w to about 2% w/w clindamycin, from about 0.05% w/w to about 0.2% w/w gentamicin, from about 1% w/w to about 3% w/w erythromycin, from about 0.1% to about 1.5% quinolone and combinations thereof.

In some embodiments, the composition of this invention comprises at least one additional active agent. In another embodiment, the additional active agent is a retinoid selected from about 0.01% w/w to about 0.5% w/w acitretin, from about 0.005% w/w to about 0.02% w/w retinol, from about 0.025% w/w to about 0.1% w/w tazarotene, from about 0.025% w/w to about 0.1% w/w tretinoin, from about 0.05% w/w to about 0.1% w/w isotretinoin and combinations thereof.

In some embodiments, the composition of this invention comprises at least one additional active agent. In another embodiment, the additional active agent is between about 1% w/w and 10% w/w benzoyl peroxide.

According to some embodiments, there is provided a composition comprising from about 0.25% w/w to about 10.0% w/w tapinarof, from about 0.05% w/ to about 20% w/w at least one additional active agent selected from a topical antibiotic, a topical retinoid, from about 1% w/w to about 20% w/w azelaic acid, from about 1% w/w to about 2% w/w clindamycin, from about 0.05% w/w to about 0.2% w/w gentamicin, from about 1% w/w to about 3% w/w erythromycin, from about 1% w/w to about 10% w/w benzoyl peroxide and combinations thereof, wherein said topical antibiotic is selected from about 1% w/w to about 2% w/w clindamycin, from about 0.05% w/w to about 0.2% w/w gentamicin, from about 1% w/w to about 3% w/w erythromycin, from about 1% w/w to about 10% w/w benzoyl peroxide, from about 0.1% to about 1.5% quinolone and combinations thereof, and a carrier suitable for topical administration.

According to some embodiments, there is provided a composition comprising from about 0.25% w/w to about 10.0% w/w tapinarof, from about 0.05% w/ to about 20% w/w at least one additional active agent selected from an antibiotic, a retinoid, from about 1% w/w to about 20% w/w azelaic acid, from about 1% w/w to about 2% w/w clindamycin, from about 0.05% w/w to about 0.2% w/w gentamicin, from about 1% w/w to about 3% w/w erythromycin, from about 1% w/w to about 10% w/w benzoyl peroxide and combinations thereof, wherein said topical antibiotic is selected from about 1% w/w to about 2% w/w clindamycin, from about 0.05% w/w to about 0.2% w/w gentamicin, from about 1% w/w to about 3% w/w erythromycin, from about 1% w/w to about 10% w/w benzoyl peroxide, from about 0.1% to about 1.5% quinolone and combinations thereof, and a carrier suitable for topical or intralesional administration.

According to some other embodiments, there is provided a composition comprising from about 0.25% w/w to about 10.0% w/w tapinarof, from about 0.05% w/ to about 20% w/w at least one additional active agent selected from a topical antibiotic, a topical retinoid, from about 1% w/w to about 20% w/w azelaic acid, from about 1% to about 10% w/w benzoyl peroxide and combinations thereof, wherein said topical retinoid is selected from about 0.01% w/w to about 0.5% w/w acitretin, from about 0.005% w/w to about 0.02% w/w retinol, from about 0.025% w/w to about 0.1% w/w tazarotene, from about 0.025% w/w to about 0.1% w/w tretinoin, from about 0.05% w/w to about 0.1% w/w isotretinoin and combinations thereof, and a carrier suitable for topical administration.

According to some other embodiments, there is provided a composition comprising from about 0.25% w/w to about 10.0% w/w tapinarof, from about 0.05% w/ to about 20% w/w at least one additional active agent selected from an antibiotic, a retinoid, from about 1% w/w to about 20% w/w azelaic acid, from about 1% to about 10% w/w benzoyl peroxide and combinations thereof, wherein said retinoid is selected from about 0.01% w/w to about 0.5% w/w acitretin, from about 0.005% w/w to about 0.02% w/w retinol, from about 0.025% w/w to about 0.1% w/w tazarotene, from about 0.025% w/w to about 0.1% w/w tretinoin, from about 0.05% w/w to about 0.1% w/w isotretinoin and combinations thereof, and a carrier suitable for topical or intralesional administration.

In some embodiments, there is provided a dosage form comprising a composition comprising from about 0.25% w/w to about 10.0% w/w tapinarof, optionally from about 0.05% w/ to about 20% w/w at least one additional active agent selected from an antibiotic, a retinoid, from about 1% w/w to about 20% w/w azelaic acid, from about 1% w/w to about 10% w/w benzoyl peroxide and combinations thereof, and a carrier for topical or intralesional administration, wherein the composition is formulated in a dosage form selected from a cream, a lotion, a gel, an ointment, an emulsion, a solution, a suspension, an elixir, a tincture, a paste, a foam, an aerosol, a spray, a patch, a transdermal patch, intralesional injection, and an applicator syringe.

In some other embodiments, there is provided a method of treatment, prevention or alleviation of hidradenitis suppurativa, comprising administering to a subject in need thereof a therapeutically effective amount of a composition comprising from about 0.25% w/w to about 10.0% w/w tapinarof, optionally from about 0.05% w/ to about 20% w/w at least one additional active agent selected from a antibiotic, a retinoid, from about 1% w/w to about 20% w/w azelaic acid, from about 1% w/w to about 10% w/w benzoyl peroxide and combinations thereof, and a carrier for topical administration, wherein the composition is formulated as a cream, a foam or as a lotion.

In another embodiment, the tapinarof and optionally the at least one active agent is/are administered topically, transdermally or intralesionally such as by intralesional injection.

In some embodiments, there is provided a method of treatment, prevention or alleviation of hidradenitis suppurativa, wherein said method comprises once daily or twice daily topical administration to a subject in need thereof of a therapeutically effective amount of the composition comprising from about 0.25% w/w to about 10.0% w/w tapinarof and a carrier suitable for topical administration.

In some embodiments, there is provided a method of treatment, prevention or alleviation of hidradenitis suppurativa, wherein said method comprises once daily or twice daily administration to a subject in need thereof of a therapeutically effective amount of the composition comprising from about 0.25% w/w to about 10.0% w/w tapinarof and a carrier, wherein the composition is administered by intralesional injection.

In some other embodiments, there is provided a method of treatment, prevention or alleviation of hidradenitis suppurativa, wherein said method comprises once daily or twice daily topical administration to a subject in need thereof of a therapeutically effective amount of the composition comprising from about 0.25% w/w to about 10.0% w/w tapinarof, from about 0.05% w/w to about 20% w/w at least one additional active agent selected from a topical antibiotic, a topical retinoid, from about 1% w/w to about 20% w/w azelaic acid, from about 1% w/w to about 10% w/w benzoyl peroxide and combinations thereof, and a carrier suitable for topical or intralesional administration.

In some other embodiments, there is provided a method of treatment, prevention or alleviation of hidradenitis suppurativa, wherein said method comprises once daily or twice daily administration to a subject in need thereof of a therapeutically effective amount of the first composition comprising from about 0.25% w/w to about 10.0% w/w tapinarof and a carrier for topical or intralesional administration, and a second composition comprising from about 0.05% w/w to about 20% w/w at least one additional active agent selected from a topical antibiotic, a topical retinoid, from about 1% w/w to about 20% w/w azelaic acid, from about 1% w/w to about 10% w/w benzoyl peroxide and combinations thereof, and a carrier for topical or intralesional administration, wherein the first and second compositions are administered concurrently or sequentially.

According to some embodiments, there is provided a method of treatment, prevention or alleviation of hidradenitis suppurativa, by topical administration to a subject in need thereof of a therapeutically effective amount of a composition comprising from about 0.25% w/w to about 10.0% w/w tapinarof, optionally from about 0.05% w/ to about 20% w/w at least one additional active agent selected from a topical antibiotic, a topical retinoid, from about 1% w/w to about 20% w/w azelaic acid, from about 1% w/w to about 10% w/w benzoyl peroxide and combinations thereof, wherein tapinarof and the at least one additional active agent exhibit an additive or synergistic effect, thereby allowing to reduce the amounts of the active agents in the composition.

According to some other embodiments, there is provided a method of treatment, prevention or alleviation of hidradenitis suppurativa, comprising administering topically or intralesionally once daily or twice daily to a subject in need thereof a therapeutically effective amount of a composition comprising from about 0.25% w/w to about 10.0% w/w tapinarof and a carrier until remission.

According to some other embodiments, there is provided a method of treatment, prevention or alleviation of hidradenitis suppurativa, comprising administering topically once daily or twice daily to a patient in need thereof a therapeutically effective amount of a composition comprising from about 0.25% w/w to about 10.0% w/w tapinarof, from about 0.05% w/ to about 20% w/w at least one additional active agent selected from a topical antibiotic, a topical retinoid, from about 1% w/w to about 20% w/w azelaic acid, from about 1% w/w to about 10% w/w benzoyl peroxide and combinations thereof, and a carrier until remission.

In some embodiments, there is provided a kit comprising one or more dosage forms comprising a therapeutically effective amount of a composition comprising from about 0.25% w/w to about 10.0% w/w tapinarof, optionally from about 0.05% w/ to about 20% w/w, from about 0.05% w/ to about 20% w/w at least one additional active agent selected from a topical antibiotic, a topical retinoid, from about 1% w/w to about 20% w/w azelaic acid, from about 1% w/w to about 10% w/w benzoyl peroxide, from about 1% w/w to about 2% w/w clindamycin, from about 0.05% w/w to about 0.2% w/w gentamicin, from about 1% w/w to about 3% w/w erythromycin, a quinolone, from about 0.01% w/w to about 0.5% w/w acitretin, from about 0.005% w/w to about 0.02% w/w retinol, from about 0.025% w/w to about 0.1% w/w tazarotene, from about 0.025% w/w to about 0.1% w/w tretinoin, from about 0.05% w/w to about 0.1% w/w isotretinoin, and combinations thereof and instructions for use.

According to some other embodiments, there is provided a method of treatment, prevention or alleviation of hidradenitis suppurativa, comprising administering once daily or twice daily to a patient in need thereof a therapeutically effective amount of a composition comprising from about 0.25% w/w to about 10.0% w/w tapinarof, from about 0.05% w/ to about 20% w/w at least one additional active agent selected from an antibiotic, a retinoid, from about 1% w/w to about 20% w/w azelaic acid, from about 1% w/w to about 10% w/w benzoyl peroxide and combinations thereof, and a carrier for topical or intralesional administration until remission.

In some embodiments, there is provided a kit comprising one or more dosage forms, for topical or intralesional administration, comprising a therapeutically effective amount of a composition comprising from about 0.25% w/w to about 10.0% w/w tapinarof, optionally from about 0.05% w/ to about 20% w/w, from about 0.05% w/ to about 20% w/w at least one additional active agent selected from an antibiotic, a retinoid, from about 1% w/w to about 20% w/w azelaic acid, from about 1% w/w to about 10% w/w benzoyl peroxide, from about 1% w/w to about 2% w/w clindamycin, from about 0.05% w/w to about 0.2% w/w gentamicin, from about 1% w/w to about 3% w/w erythromycin, a quinolone, from about 0.01% w/w to about 0.5% w/w acitretin, from about 0.005% w/w to about 0.02% w/w retinol, from about 0.025% w/w to about 0.1% w/w tazarotene, from about 0.025% w/w to about 0.1% w/w tretinoin, from about 0.05% w/w to about 0.1% w/w isotretinoin, and combinations thereof and instructions for use.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or” treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

As used herein, the term “essentially free” generally refers to a composition having less than about 2 percent by weight, more preferably 1 percent per weight, less than about 0.5 percent by weight or even less than 0.1 percent by weight of a certain ingredient, based on the total weight of the composition.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader. All the information contained therein is incorporated herein by reference.

### Example 1

Preparation of a Tapinarof Cream Composition

The topical tapinarof cream consists of:

0.25-2.0% w/w tapinarof,

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

6.0-7.0% of 0.1M NaOH or HCl as an aqueous phase pH.

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof cream in an aluminum tube or other delivery system.

### Example 2

Preparation of a Tapinarof-Benzoyl peroxide Cream Composition

The topical tapinarof-benzoyl peroxide combination cream consists of:

0.25-2.0% w/w tapinarof,

1-10% w/w encapsulated benzoyl peroxide,

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

6.0-7.0% of 0.1M NaOH or HCl as an aqueous phase pH.

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, benzoyl peroxide, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof-benzoyl peroxide combination cream in an aluminum tube or other delivery system.

### Example 3

Preparation of tapinarof, 1% lotion

| Raw Material (compendial Name) | % w/w |
|---|---|
| Tapinarof 98% | 1.0 |
| Castor oil | 4.0 |
| Mineral oil light | 4.0 |
| Diethylene glycol monoethyl ether | 5.5 |
| Dimethyl Sulfoxide | 5.5 |
| Sorbitan Monooleate | 0.1 |
| Propylene glycol | 10.0 |
| Disodium Edetate (EDTA) | 0.1 |
| Carbomer Copolymer Type B Pemulen ®TR-1 | 0.4 |
| Carbomer Homopolymer Type A Carbopol ®981 | 0.6 |
| Purified water | 64.00 |
| Benzoic Acid | 0.25 |
| BHT | 0.1 |
| Citric Acid | 0.1 |
| Sodium Citrate | 0.2 |
| Sodium hydroxide pellets | For pH adjustment |
| Purified water | q.s. to 100 |

### Water Phase

Into a glass beaker water and Benzoic Acid were added. The beaker was placed inside a hot water bath adjusted to 60° C. and the mixture was mixed with a magnetic stirrer until a clear solution free from particles was obtained. Then EDTA, Citric Acid and Sodium Citrate were added. The mixing was continued until a clear solution was obtained. The solution was cooled down to room temperature. Then, the pH was slowly adjusted to pH 6.0 with NaOH 20%.

### Oil Phase

In a separate glass beaker Mineral oil light, castor oil, span 80 and BHT were weighed. The beaker was placed inside a hot water bath adjusted to 60° C. and the mixture was mixed with a magnetic stirrer until a uniform solution was obtained. Then Carbopol®981 and Pemulen®TR-1 were slowly added and the mixing was continued until a homogenous mixture was obtained. The mixture was cooled down to room temperature.

### Active Phase

Into a separate glass beaker Propylene Glycol, Transcutol and DMSO were weighed. The mixture was mixed with a magnetic stirrer until a uniform homogenous solution was obtained. The beaker was covered with an aluminum foil and placed in a yellow light hood. Tapinarof was slowly added, and the mixing was continued for about 1 h until a clear solution free from particles was obtained.

The oil phase was slowly added to the water phase while homogenizing for about 5 minutes, until there were no lumps. Then, the active phase was slowly added to the Water+Oil phase while homogenizing for about 5 minutes.

Water was added for batch completion, and final pH was measured to conform it is around pH 5.

1. A method of treatment, prevention and/or amelioration hidradenitis suppurativa, comprising administering a composition comprising from about 0.25% w/w to about 10.0% w/w tapinarof and a carrier.

2. The method of claim 1, wherein the composition is formulated for topical, transdermal or intralesional administration.

3. The method of claim 1, wherein the composition further comprising from about 0.05% w/w to about 20% w/w at least one additional active agent selected from an antibiotic, a retinoid, azelaic acid, benzoyl peroxide and combinations thereof, and a suitable carrier.

4. The method of claim 3, wherein said antibiotic is selected from about 1% w/w to about 2% w/w clindamycin, from about 0.05% w/w to about 0.2% w/w gentamicin, and from about 1% w/w to about 3% w/w erythromycin, and from about 0.1% to about 1.5% quinolone and combinations thereof.

5. The method of claim 3, wherein said retinoid is selected from about 0.01% w/w to about 0.5% w/w acitretin, from about 0.005% w/w to about 0.02% w/w retinol, from about 0.025% w/w to about 0.1% w/w tazarotene, from about 0.025% w/w to about 0.1% w/w tretinoin, from about 0.05% w/w to about 0.1% w/w isotretinoin and combinations thereof.

6. The method of claim 3, wherein the composition comprises between about 1% w/w and 10% w/w benzoyl peroxide.

7. A dosage form comprising a composition comprising from about 0.25% w/w to about 10.0% w/w tapinarof and a carrier wherein the composition is for use in the of treatment, prevention and/or amelioration of hidradenitis suppurativa and is formulated in a dosage form selected from a cream, a lotion, a gel, an ointment, an emulsion, a solution, a suspension, an elixir, a tincture, a paste, a foam, intralesional injection, an aerosol, a spray, a patch, a transdermal patch and an applicator syringe.

8. (canceled)

9. The method of claim 1, wherein the composition is administered topically and is formulated as a cream, a foam or as a lotion.

10. The method of claim 1, wherein the treatment comprises once daily or twice daily topical administration to a subject in need thereof of a therapeutically effective amount of said composition.

11. The method of claim 3, wherein tapinarof and the at least one additional active agent exhibit an additive or synergistic effect, thereby allowing to reduce the amounts of the active agents in the composition.

12. A regimen of administering a composition comprising from about 0.25% w/w to about 10.0% w/w tapinarof and a carrier for use in the of treatment, prevention and/or amelioration of hidradenitis suppurativa; wherein the administration comprises once daily or twice daily administration to a patient in need thereof of a therapeutically effective amount of the composition until remission.

13. A regimen of administering a composition comprising from about 0.25% w/w to about 10.0% w/w tapinarof and from about 0.05% w/w to about 20% w/w at least one additional active agent selected from an antibiotic, a retinoid, azelaic acid, benzoyl peroxide and combinations thereof, and a suitable carrier for use in the of treatment, prevention and/or amelioration of hidradenitis suppurativa; wherein the administration comprises the once daily or twice daily administration a patient in need thereof a therapeutically effective amount of the composition until remission.

14. A kit comprising one or more dosage forms of claim 7 and instructions for use.

15. The method of claim 2, wherein the composition further comprising from about 0.05% w/w to about 20% w/w at least one additional active agent selected from an antibiotic, a retinoid, azelaic acid, benzoyl peroxide and combinations thereof, and a suitable carrier.
